(19) United States
(12) Patent Application Publication
(10) Pub. No.: US 2022/0354238 A1
(43) Pub. Date: Nov. 10, 2022

(54) METHOD FOR DESIGNING COSMETIC PRODUCTS WITH IMPROVED SUSTAINABILITY BENEFITS

(71) Applicant: ELC MANAGEMENT LLC, Melville, NY (US)

(72) Inventors: MaryAnn McKeever-Alfieri, East Islip, NY (US); Paul Scott, Pelham, NY (US); George C. Daher, Pleasant Prairie, WI (US); Eva Claire Thompson, Astoria, NY (US); Julie Beth Zimmerman, Guilford, CT (US); Paul Thomas Anastas, Guilford, CT (US); Matthew Jensen Eckelman, Roslindale, MA (US); Matthew S. Moroney, Somerville, MA (US)

(21) Appl. No.: 17/735,044

(22) Filed: May 2, 2022

Related U.S. Application Data
(60) Provisional application No. 63/183,618, filed on May 3, 2021.

(51) Int. Cl. A45D 44/00

(52) U.S. Cl. CPC A45D 44/005

(57) ABSTRACT

The present invention relates to a method for identifying ingredients or compositions, based on green chemistry performance, comprising the steps of: (a) identifying a cosmetic ingredient or a cosmetic composition; (b) calculating each of; (b1) a Green Chemistry Performance Score; (b2) a Binary Score; and (b3) a Certainty Score; and (c) selecting the cosmetic ingredient or the cosmetic composition having an aggregate score above the category average.

### FIELD OF THE INVENTION

The present invention relates to methods for assessing and generating cosmetic products with improved environmental impact and sustainability attributes.

### BACKGROUND OF THE INVENTION

Efforts to design cosmetic products which have reduced environmental impact are often challenged by various tradeoffs. For example, ingredients and formulas with improved environmental impact profiles may suffer from reductions on product performance, human health considerations, or shelf-life limitations. Also, ingredient combinations may create residual effects which are difficult to predict. Maintaining up-to-date information regarding ingredients and formulations is also hampered by the ongoing emergence of new ingredients and ingredient combinations. There remains a need for a structured system which is capable of predictably assessing and generating cosmetic products with improved environment impact and sustainability attributes.

### SUMMARY OF THE INVENTION

The present invention relates to a scoring system which enables efficient selection of ingredients, or combinations of ingredients, with a high degree of confidence that such selections will result in strong green chemistry performance. The scoring system is defined by at least the following distinct elements: (1) a performance Score, (2) a Binary Score, and (3) a Certainty Score; and

### BRIEF DESCRIPTION OF THE FIGURES

FIG. 1 shows exemplary criteria which may contribute to assessing a Human Health score.

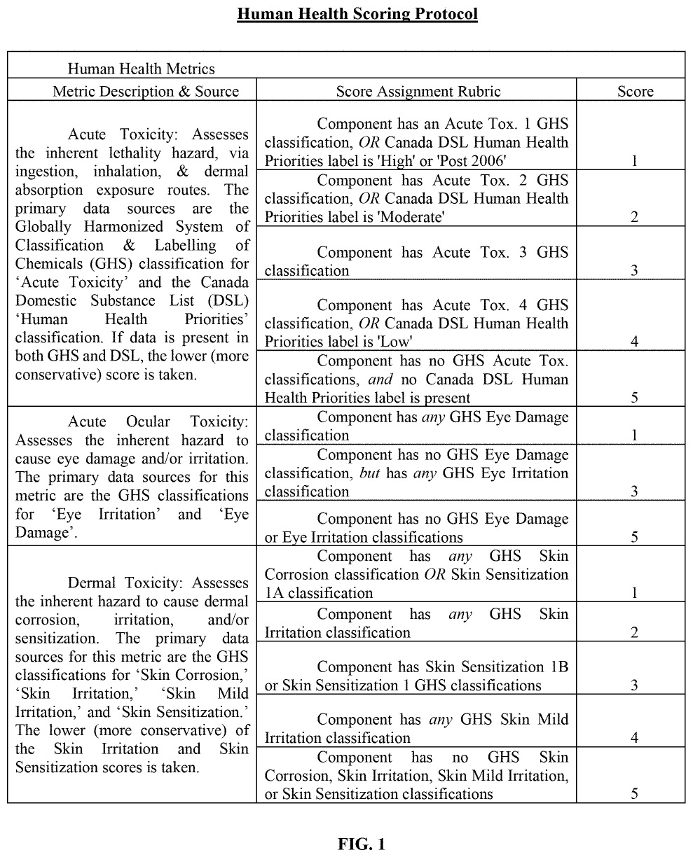

FIG. 2 shows exemplary criteria which may contribute to assessing an Ecosystem Health score.

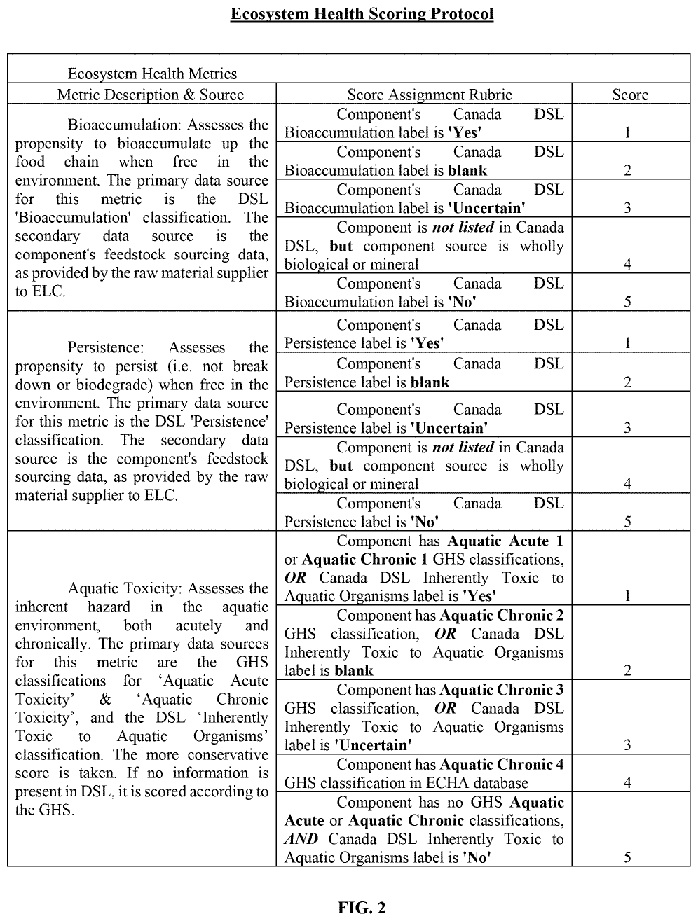

FIG. 3 shows exemplary criteria which may contribute to assessing an Environment score.

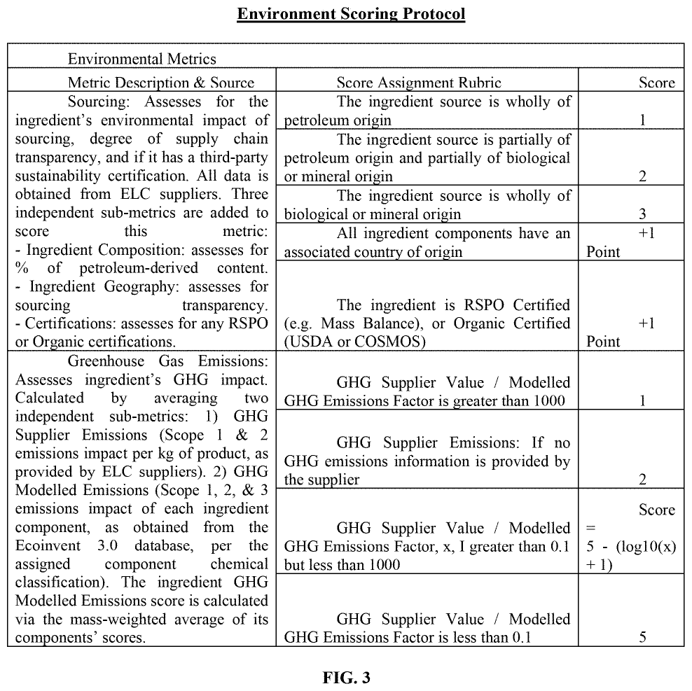

FIG. 4 shows exemplary default scores which may be used as proxies for certain ingredient classes.

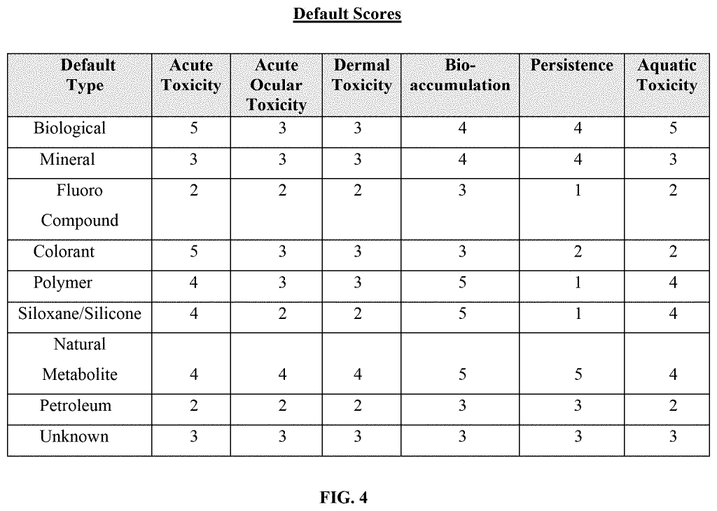

FIG. 5 shows exemplary criteria which may contribute to assessing an Certainty score.

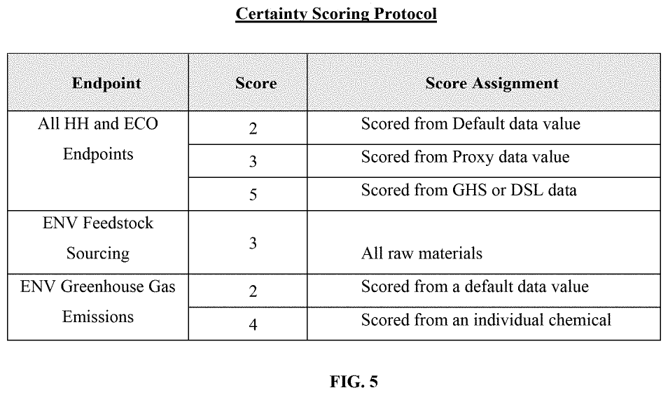

### DETAILED DESCRIPTION OF THE INVENTION

Performance Score

The first component of the scoring system assesses ingredient or formula green chemistry performance across three categories, including:

1. Human Health (HH)

2. Ecosystem Health (ECO)

3. Environment (ENV)

The performance score is calculated according to the following formula:

$$Green Chemistry performance score = \frac{H H + E C O + E N V}{3}$$

Each element (HH, ECO, ENV) of an ingredient's green chemistry performance score is obtained calculating the average of equally weighted data points specific to that element. According to the present scoring system, the calculation comprises eight individual metrics; three for Human Health, three for Ecosystem Health and two for Environment. Each of the aforementioned metrics are calculated according to the following equations:

$$Human Health ( HH ) = \frac{\begin{matrix}Acute Toxicity + Acute Ocular Toxicity + \\ Acute Dermal Toxicity\end{matrix}}{3}$$

$$Ecosystem Health ( ECO ) = \frac{\begin{matrix}Bioaccu m u l a t i o n + P e r s i s t e n ce + \\ Aquatic Toxicity\end{matrix}}{3}$$

$$Environment ( ENV ) = \frac{Feed stock Sourcing + GHG Emissions}{2}$$

### Individual Metrics for Each Category

I. Each of the measurements may be initially scored on a 1-5 scale, then converted to a 0-100 scale to simplify for the end user and allow for enhanced interpretation and ease of use. The data sources should be normalized to provide consistent and reliable scoring outcomes. Water is preferably excluded from the ingredient scoring system and metric calculations; as such, it is not considered a component of any ingredient for the purposes of the scoring process. Because the scoring system is a hazard-based assessment tool, and water has essentially zero hazard in personal care ingredients and products, its exclusion from the scoring system calculations ensures that focus is placed on the presence of known or potentially hazardous ingredient components. It also ensures that the positive or negative Human Health, Ecosystem Health (ECO) and Environmental (ENV) contributions of each component are highlighted. Human Health (HH)

The Human Health (HH) score comprises 3 variable components which conform to the following equations:

Human Health (HH)=(Acute Toxicity+Acute Ocular Toxicity+Dermal Toxicity)/3

The Human Health score is calculated by analyzing the chemical components of each assessed ingredient for each of the following factors:

### Acute Toxicity

Acute Toxicity assesses the inherent lethality hazard of the component, through ingestion, inhalation, and dermal absorption routes of exposure. The data source for this metric is the Globally Harmonized System of Classification and Labeling of Chemicals (GHS) classification for ‘Acute Toxicity’ and the Canada Domestic Substance List (DSL) ‘Human Health Priorities’ classification.

Acute Toxicity is measured according to the following equation: Σᵢ₌₁ⁿATᵢ*wᵢ, where: ATᵢ=the Acute Human Toxicity score of each component, wᵢ=the proportion, by mass, of each component in the ingredient (after water has been removed as a component), and n =the number of components in the ingredient.

### Acute Ocular Toxicity

Acute Ocular Toxicity assesses the inherent hazard of the component to cause eye damage and/or irritation. The primary data sources for this metric are the GHS classifications for ‘Eye Irritation’ and ‘Eye Damage’.

Acute Ocular Toxicity is measured according to the following equation: Σᵢ₌₁ⁿOCᵢ*wᵢ, where: OCᵢ=the Acute Ocular Toxicity score of each component, wᵢ=the proportion, by mass, of each component in the ingredient (after water has been removed as a component), and n=the number of components in the ingredient.

### Dermal Toxicity

Dermal Toxicity assesses the inherent hazard of the component to cause dermal corrosion, irritation, and/or sensitization. The primary data sources for this metric are the GHS classifications for ‘Skin Corrosion,’ ‘Skin Irritation,’ and ‘Skin Mild Irritation’ (for Skin Irritation sub-metric), and ‘Skin Sensitization’ (for Skin Sensitization sub-metric).

Dermal Toxicity is measured according to the following equation: Σᵢ₌₁ⁿDᵢ* wᵢ, where: Dᵢ=the Dermal Toxicity score of each component, wᵢ =the proportion, by mass, of each component in the ingredient (after water has been removed as a component), and n=the number of components in the ingredient.

The preliminary (pre-penalty and pre-scaling) Acute Toxicity, Acute Ocular Toxicity, and Dermal Toxicity metric scores for each ingredient are obtained by calculating the mass-weighted averages of its components' metric scores.

For example, Acute Ocular Toxicity=Σᵢ₌₁ⁿAᵢ*wᵢ, where: n=the number of components in the ingredient, Aᵢ=the Acute Ocular Toxicity score of each component, and wᵢ=the proportion, by mass, of each component in the ingredient

The preliminary (pre-scaling) Human Health score for each ingredient is then obtained by averaging the ingredient's scores for the three Human Health metrics. That is:

Human Health (HH)=(Acute Toxicity+Acute Ocular Toxicity+Dermal Toxicity)/ 3

### II. Ecosystem Health

Ecosystem Health is calculated by analyzing the chemical components of each ingredient for each of the following factors:

### Bioaccumulation

Bioaccumulation assesses the propensity of the component to bioaccumulate up the food chain when free in the environment. The data source for this metric is the component's feedstock sourcing data and the DSL ‘Bioaccumulation’ classification.

Bioaccumulation is measured according to the following formula: Σᵢ₌₁ⁿBᵢ* wᵢ, where: Bᵢ=the Bioaccumulation score of each component, wᵢ=the proportion, by mass, of each component in the ingredient (after water has been removed as a component), and n=the number of components in the ingredient.

### Persistence

Persistence assesses the propensity of the component to persist (i.e. not break down or biodegrade) when free in the environment. The data source for this metric is the component's feedstock sourcing data and the DSL ‘Persistence’ classification.

Persistence: Σᵢ₌₁ⁿPᵢ*ᵢ, where: Pᵢ=the Persistence score of each component, wᵢ=the proportion, by mass, of each component in the ingredient (after water has been removed as a component), and n=the number of components in the ingredient.

### Aquatic Toxicity

Aquatic Toxicity assesses the inherent hazard of the component in the aquatic environment, both acutely and chronically. The primary data sources for this metric are the GHS classifications for ‘Aquatic Acute Toxicity’ and ‘Aquatic Chronic Toxicity’, along with the DSL ‘Inherently Toxic to Aquatic Organisms’ classification. Aquatic Toxicity: Σᵢ₌₁ⁿAQᵢ*wᵢ, where: AQᵢ=the Aquatic Toxicity score of each component, wᵢ=the proportion, by mass, of each component in the ingredient (after water has been removed as a component), and n=the number of components in the ingredient.

The preliminary (pre-penalty and pre-scaling) Bioaccumulation, Persistence, and Aquatic Toxicity metric scores for each ingredient are obtained by calculating the mass-weighted averages of its components' metric scores.

For example, Persistence=Σᵢ₌₁ⁿP₁*wᵢ, where: n=the number of components in the ingredient, Pᵢ=the Persistence score of each component, and wᵢ=the proportion, by mass, of each component in the ingredient.

The preliminary (pre-scaling) Ecosystem Health score for each ingredient is then obtained by averaging the ingredient's scores for the three Ecosystem Health metrics. That is:

$$Ecosystem Health ( ECO ) = \frac{\begin{matrix}Bioaccu m u l a t i o n + P e r s i s t e n ce + \\ Aquatic Toxicity\end{matrix}}{3}$$

### III. Environment

Environmental Impact is calculated by analyzing each ingredient for each of the following factors:

### Feedstock Sourcing

The Feedstock Sourcing value assesses the environmental impact of the sourcing of each ingredient, along with the degree of transparency of the ingredient's supply chain, and if the ingredient has any third party-certified sustainability benefits. Three independent sub-metrics are added to score this metric:
Ingredient Composition assesses the ingredient's percentage of petroleum-based content. The data source for this metric is the raw material's feedstock sourcing data, as provided by the raw material supplier to the user.
Ingredient Geography assesses the transparency of sourcing for each of the ingredient's components. The data source for this metric is the raw material's feedstock sourcing data, as provided by the raw material supplier to the user.
Certifications assesses the if the ingredient has any third-party sustainability certifications. Currently, this includes RSPO and Organic certifications. Eligible certifications for this sub-metric can be updated and evolved based on business and consumer preference. The data source for this metric is the raw material's certification status, as provided by the raw material supplier to the user.

### GHG Emissions

Greenhouse gas (GHG) emissions are assessed for impact of each ingredient. It is calculated by averaging two independent sub-metrics of GHG Supplier Emissions and GHG Modelled Emissions. The calculation method and data sources for this metric can be updated and evolved to reflect improved GHG assessment methodologies and emerging science.

GHG Supplier Emissions assesses the Scope 1 & 2 GHG emissions impact of each ingredient. The data source for this metric may be an end-user-conducted GHG supplier emissions survey.

GHG Modelled Emissions assesses each ingredient component's Scope 1, 2 & 3 GHG emissions impact. The data source for this metric is the Ecoinvent 3.0 life cycle inventory database (www.ecoinvent.org). The ingredient's GHG Modelled Emissions score is then obtained by calculating the mass-weighted average of its component's scores.

The preliminary (pre-penalty and pre-scaling) Environment score for each ingredient is then obtained by averaging the ingredient's scores for the two Environment metrics. That is:

$$Environment ( ENV ) = \frac{Feed stock Sourcing + GHG Emissions}{2}$$

Any applicable penalty score is then deducted from the ingredient's Environment score. More information on how penalty scores are calculated can be found in the Penalty Score for Numeric Value section of this document.

### Penalty Score for Numeric Value

The scoring system is designed to drive formulator behavior towards green chemistry. In order to send a clear message away from ingredients that score poorly, the tool includes a penalty system that amplifies the impact of the lowest scores. Penalties can be reviewed as required, for example if new scientific data becomes available. Increasing the penalty value for components and ingredients could be used to increase ambition, or refine approved chemical inventories, for example.

In the current system, a penalty of 0.1 is applied for every data point (within the scoring of the eight individual metrics) that receives a score of “1” (the lowest score on our scale), the total penalty is then deducted from the total score to give the final (penalty adjusted) score.

The penalty scores for the HH and ECO metrics occur at the component level, but the penalty score for the ENV metrics occurs at the ingredient level. A score of 0 is assigned if penalties would bring the scoring system to a negative number.

### Binary Score

The Second element of our scoring tool is the functionality to tag ingredients based on characteristics that are particularly important for consumer and regulatory environment within which the beauty and personal care industry operates in and contributes to. As such, each ingredient is screened for ten binary metrics that can be assigned as tags/indicators. Indicators 1-6 are determined before scoring based on checks against across supply chain data sources. Indicators 7-10 are determined once the components and raw materials have been scored using the green score tool.

| Indicator | Description | |
|---|---|---|
| Administrative Ban | This may be assigned to a decide to ban the use of an | |
| | ingredient following a regulatory assessment. | |
| Carcinogenic, | This tag addresses materials that are known or potential | |
| Mutagenic, and/or | carcinogens. An ingredient receives the tag if any of its | |
| Reproductive Toxicity | components are included on the following lists; | |
| (CMR) | CMR substances of category 1A, 1B or 2 under | |
| | European Commission No 790/2009 & 1272/2008.1 | |
| | EU Cosmetics Regulation Annex 2 | |
| | ECHA Annex 6 CPL List | |
| Endocrine Disruption | This tag addresses materials with known or potential | |
| (ED) | human endocrine disruption properties. An ingredient receives | |
| | the tag if any of its components are included on the following | |
| | lists; | |
| | 1. | European Commission, substances suspected of |
| | | interfering with the hormone systems (COM (1999) |
| | | 706). (COM (2001) 262) and (SEC (2004) 1372) |
| | 2. | UN List of Identified Endocrine Disrupting Compounds |
| | | Overview Report II-IPCP (July 2017) |
| Dermal Allergenicity | This tag addresses materials that are known skin | |
| | allergens. An ingredient receives the tag if any of its | |
| | components are on the “list of allergenic compounds” from the | |
| | following sources: | |
| | 1. | Chapter IV. Sensitizing Substances, in ‘List of MAK and |
| | | BAT Values 2014. DFG, Deutsche |
| | | Forschungsgemeinschaft’ |
| | 2. | European Commission, Scientific Committee on |
| | | Perfume Allergies, |
| | | https://ec.europa.eu/health/scientific_committees/ |
| | | opinions_layman/perfume-allergies/en/1-3/1-introduction.htm |
| | 3. | haz-map, Table 1--Chemicals that Cause Occupational |
| | | Allergic Contact Dermatitis. |
| | | https://www.haz-map.com/allergic.htm |

| | |
|---|---|
| NGO List of Concern | This tag addresses materials that are of concern to |
| (NGO) | NGOs, specifically EWG. An ingredient receives the tag if any |
| | of its components are included in the EWG Skin Deep Database |
| | and score between 3-10. |
| Potential Impurities | This tag addresses materials that have potential |
| | impurities as a result of their manufacturing process, |
| | specifically 1,4-dioxane. An ingredient receives the tag based |
| | on information provided to a company from its suppliers |
| | regarding potential presence of 1,4-dioxane. |
| Animal-derived | This tag addresses materials that are animal derived or |
| | made with animal derived processing aids. An ingredient |
| | receives the tag based on information provided to a company |
| | from its suppliers regarding whether ingredients are derived |
| | from animal sources or processed with animal-derived |
| | materials. |
| Environmentally | This tag addresses materials that are ( ) environmentally |
| Persistent, Bioaccumulative, | persistent, bioaccumulative, and toxic. An ingredient receives |
| & Toxic (PBT) | the tag if any of its components score a 1 on all three of the ECO |
| | metrics (pre-scaling). |
| Inherently | This tag addresses materials that are (inherently |
| Biodegradable | biodegradable,. An ingredient receives the tag if its Persistence |
| | metric score is ≥4 (pre-scaling). |

### Certainty Score

The scoring system herein is calculated using multiple datapoints to provide an accurate assessment of the green chemistry performance for each ingredient and formula. For each metric, we seek to use the best available method of assessment and data—driven by the latest science. However, it is essential to recognize that there is variation in scientific knowledge, scientific methods and data sets available. For example, the body of evidence for human health metric acute toxicity is very different from that of the Environment Metric Greenhouse Gas Emissions (GHGs)—in short, they are at different stages of scientific discovery. Our approach recognizes and responds to this through the “Certainty Score” assigned to each data point at each stage of our calculations.

Each metric and category score for each ingredient and ingredient component, along with the ingredient's Numeric Value, has an associated Certainty Score that indicates the robustness of each metric's data source, and reflects the complexity and evolution of the science and scientific models underlying the scoring. As such, they are analogous to the error bars often seen in scientific research. The Certainty Score thus gives the user insight into the quality and robustness of the science underlying the data, and as such, informs their decision-making process. The Certainty Score is most relevant at the metric level and is directional at the ingredient level. We calculated a Certainty Score at the formula level but do not use this to inform decision making.

The calculation function of the Certainty Score is done with exactly the same equations as the numerical value associated with the green score—however, in order to avoid confusion, the Certainty Score remains on a 1-5 scale (and as such does is not scaled to a 0-100 scale).

The Certainty Scores for the HH and ECO metrics, along with the GHG Modeled Emissions sub-metric occur at the component level, and a mass-weighted average is calculated to obtain the ingredient-level Certainty Score for those ingredients with multiple components. The Certainty Score for the Sourcing metric and the GHG Supplier Emissions sub-metric occurs at the ingredient level.

### Calculating Green Score at the Formula Level

### Green Score Numeric Value

The formula Green Score calculation is separate from the ingredient calculation. Formula Green Scores are calculated by summing the mass weighted ingredient green score numeric values, as shown below. Equivalent mathematical operations are used to calculate the formula's high-level HH, ECO, and ENV scores, along with all of the formula's individual metric scores (e.g. Acute Ocular Toxicity, Bioaccumulation).

$$Formula Green Score = {\sum }_{i = 1}^{n} {GS}_{i} * \frac{{P}_{i}}{100 * ( 1 - ( \frac{w}{100} ) )} ,$$

where GSᵢ=the Green Score Numeric Value of each ingredient, Pᵢ=the percentage of the ingredient in the formula, w=the percentage of water in the formula (NOT including water content of specific ingredients, only that added to the formula), and n=the number of ingredients in the formula. As with the ingredient Green Score calculations, water is excluded from the formula Green Score calculation. Excluding water from the Green Score calculation ensures that focus is placed on ingredient choice, and the presence of known or potentially hazardous materials in formula, rather than chemical dilution. It also ensures that the positive or negative HH, ECO, and ENV contributions of each raw material are highlighted.

### Green Score Use Case

The Green Score tool data enables a user to integrate a quantifiable metric of green chemistry performance into the creation of all formulas across all product categories. This further enables the user to establish data-driven, trackable green chemistry performance benchmarks for each product category and sub-category. The established category and sub-category benchmarks are then used to inform new product launches, drive innovation, and track our commitment to green chemistry performance across the entire product portfolio. Initially, these benchmarks will be internal metrics; however, over time we will seek to share them with our stakeholders.

### Product Category Benchmarks

The product category benchmarks are calculated by grouping all active formulas for each of a user's cosmetic product categories and sub-categories (e.g., categories: haircare, skincare, make-up, fragrance; sub-categories: serums, waterproof mascara, conditioners, solid perfumes). The equation for product category and sub-category benchmark calculation is as follows:

$$Green Score Benchmark = \frac{{\sum }_{i = 1}^{n} F {S}_{i}}{n} ,$$

where: FSᵢ=the Formula Green Score Numeric Value of each active product in a given category/sub-category, and n=the number of products in the category/sub-category

The lowest and highest scoring formulas in each product category and sub-category are also highlighted to enhance understanding of the range of potential scores for the particular category/sub-category.

The product category and sub-category benchmarks can be adjusted as needed, such as to make formulation green chemistry requirements stricter. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for identifying ingredients or compositions, based on green chemistry performance, comprising the steps of:
(a) identifying a cosmetic ingredient or a cosmetic composition;
(b) calculating each of;
(b1) a Green Chemistry Performance Score;
(b2) a Binary Score; and
(b3) a Certainty Score; and
(c) selecting the cosmetic ingredient or the cosmetic composition having an aggregate score above the category average.

2. A method according to claim 1, wherein each of said scores is not impacted by said ingredient's or said composition's water content.

3. A method according to claim 1, wherein said Green Chemistry Performance Score comprises an (average, sum) of sub-scores selected from;
a. Human Health includes; Acute Toxicity (AT), Acute Ocular Toxicity (AOT), and Dermal Toxicity (DT);
b. Ecosystem Health includes:
i. Bioaccumulation (B), Persistence (P), Aquatic Toxicity (AqT)3;
c. Environment includes:
i. Feedstock Sourcing (FS) & Greenhouse Gas Emissions (GHG).

4. A method according to claim 1, wherein calculating said Certainty Score comprises the steps of:
a. summing the mass weighted ingredient green scores;
b. equivalent mathematical operations are used to calculate the formula's high-level HH, ECO, and ENV scores, along with all of the formula's individual metric scores;
c. a tag of each of the ingredients are collated and presented at the formula level;
d. calculating product category benchmarks by grouping all active formulas for each product category and sub-category (e.g., categories: haircare, skincare, makeup, fragrance; sub-categories: serums, waterproof mascara, conditioners, solid perfumes);
e. benchmarks are set as the mean value of each category and sub-category; and
f. calculating the lowest and highest scoring formulas in each product category and sub-category are also highlighted to enhance understanding of the range of potential scores for the particular category/sub-category.
